(19) United States
(12) Patent Application Publication
(10) Pub. No.: US 2026/0192021 A1
(43) Pub. Date: Jul. 9, 2026

(54) COMPOSITE, PRODUCTION METHOD THEREOF AND BONE REGENERATION MATERIAL

(71) Applicants: OSAKA DENTAL UNIVERSITY, Osaka-shi, Osaka (JP); NATIONAL UNIVERSITY CORPORATION KYOTO INSTITUTE OF TECHNOLOGY, Kyoto-shi, Kyoto (JP); TOAGOSEI CO., LTD., Minato-ku, Tokyo (JP)

(72) Inventors: Yoshitomo Honda, Hirakata-shi, Osaka (JP); Tomonari Tanaka, Kyoto-shi, Kyoto (JP)

(73) Assignees: OSAKA DENTAL UNIVERSITY, Osaka-shi, Osaka (JP); NATIONAL UNIVERSITY CORPORATION KYOTO INSTITUTE OF TECHNOLOGY, Kyoto-shi, Kyoto (JP); TOAGOSEI CO., LTD., Minato-ku, Tokyo (JP)

(21) Appl. No.: 18/729,965

(22) PCT Filed: Jan. 19, 2023
(86) PCT No.: PCT/JP2023/001465

§ 371 Date: Jul. 18, 2024

(30) Foreign Application Priority Data
Jan. 21, 2022 (JP) 2022-007576

(51) Int. Cl. A61L 27/46; A61L 27/22; A61L 27/52; A61L 27/54

(52) U.S. Cl. CPC A61L 27/46; A61L 27/222; A61L 27/52; A61L 27/54; A61L 2430/02

(57) ABSTRACT

Provided are a material with high bone regeneration efficiency and a method for producing the material. A composite containing a matrix and β-tricalcium phosphate (β-TCP) particles dispersed in the matrix, and the matrix contains a functional crosslinked structure wherein a natural polymer having at least one member selected from the group consisting of an OH group and an amino group and a COOH group in a repeating unit is bound to a physiologically active substance having an OH group or an amino group via an ester bond, an amide bond, or a hydrogen bond, and the COOH group of the natural polymer is crosslinked with the at least one member selected from the group consisting of an OH group and an amino group of the natural polymer.

### TECHNICAL FIELD

The present invention relates to a composite, a production method for the composite, and a bone regeneration material.

### BACKGROUND ART

β-tricalcium phosphate (β-TCP) particles (NPL 1 to 3) and a spongy material made of a combination of β-TCP particles and a polymer (NPL 4) have been used as bone regeneration materials.

PTL 1 discloses a functional crosslinked structure composed of a physiologically active substance (e.g., catechins) conjugated with a crosslinked natural polymer. PTL 2 discloses the application of a composite of catechins and gelatin in a bone destruction inhibitor.

### CITATION LIST

### Non-Patent Literature

NPL 1: Mater. Today Bio 2019, 4, 100031.
NPL 2: Biomater. Res. 2019, 23, 12.
NPL 3: Acta Biomater. 2021, 126, 496-510.
NPL 4: Polymers 2019, 11, 1468.

### PATENT LITERATURE

PTL 1: JP2015-208369A
PTL 2: JP2018-165248A

### SUMMARY OF INVENTION

### Technical Problem

An object of the present invention is to provide a composite, a production method for the composite, and a bone regeneration material.

### Solution to Problem

The present invention provides the following composite, method for producing the composite, and bone regeneration material.

[1] A composite comprising
a matrix, and
β-tricalcium phosphate (F-TCP) particles dispersed in the matrix,
the matrix comprising a functional crosslinked structure wherein
a natural polymer having at least one member selected from the group consisting of an OH group and an amino group, and a COOH group in a repeating unit is bound to a physiologically active substance having an OH group or an amino group via an ester bond, an amide bond, or a hydrogen bond, and
the COOH group of the natural polymer is crosslinked with the at least one member selected from the group consisting of an OH group and an amino group of the natural polymer.
   [2] The composite according to [1], wherein the physiologically active substance is at least one member selected from the group consisting of catechins, polyphenols, proanthocyanidins, anthocyanins, flavonoids, soy isoflavones, and polyamines.
   [3] The composite according to [1] or [2], wherein the natural polymer is selected from the group consisting of hyaluronic acid, collagen, gelatin, xanthan gum, gellan gum, pectin, pectic acid, alginic acid, a sodium salt of alginic acid, and elastin.
   [4] The composite according to any one of [1] to [3], wherein the matrix is a gel.
   [5] The complex according to any one of [1] to [4],
wherein the physiologically active substance is a catechin that mainly contains epigallocatechin gallate (EGCG), and
the natural polymer is gelatin.
   [6] A method for producing a composite of a functional crosslinked structure and β-TCP particles,
the method comprising
reacting a natural polymer having at least one member selected from the group consisting of an OH group and an amino group, and a COOH group in a repeating unit with a physiologically active substance having an OH group or an amino group,
in water, and in the presence of a water-soluble dehydration-condensation agent,
to bind the COOH group of the natural polymer to the physiologically active substance via an ester bond, an amide bond, or a hydrogen bond, and
to crosslink the natural polymer,
thereby obtaining a functional crosslinked structure, and
mixing a solution of the obtained functional crosslinked structure with D-tricalcium phosphate (β-TCP) particles, and
subjecting the mixture to lyophilization and vacuum heating.
   [7] The method according to [6], further allowing a water-containing solution to the obtained composite
to act to obtain a gelled composite.
   [8] A bone regeneration material comprising the composite of any one of [1] to [5].

### Advantageous Effects of Invention

The functional crosslinked structure, which contains a crosslinked natural polymer bound to a physiologically active substance, and β-TCP particles, are both constituents of the composite of the present invention and are known substances.

It is, however, considered to be an unexpected result that a combination of these components dramatically increases bone regeneration capacity and operability.

### BRIEF DESCRIPTION OF DRAWINGS

FIG. 1 is a schematic view showing a method for producing a sponge, which is a precursor of a hydrogel.

FIG. 2 characterizes sponges that are precursors of hydrogels. (A) Macroscopic appearance of sponges. (B) Microscopic appearance of sponges. The arrows indicate β-tricalcium phosphate (β-TCP) particles. (C) Spectra of attenuated total reflection Fourier transform infrared spectroscopy (ATR-FTIR). (D) X-ray powder diffraction (XRD) results for materials.

FIG. 3 shows microscopic images of β-TCP leakage and hydrogels. (A) Typical macroscopic images of hydrogels and quantitative data after immersion in water. Arrows: Leaked β-TCP particles. (B) Microphotographs of hydrogels after gelatin staining.

FIG. 4 shows hydrogel morphologies and water absorption rates. (A) Hydrogel morphologies after application of ultrapure water to sponges. (B) Water absorption rates. * p<0.05, ** p<0.01, n.s.: not significant.

FIG. 5 shows the biocompatibility of hydrogels. (A) Cytotoxicity of hydrogels tested in CCK-8 assay and osteoblast cell line UMR106 (n=3) after 24 to 72 hours of culture. (B) Stained live osteoblast cell line UMR106 and stained dead osteoblast cell line UMR106 after 24 hours of culture. Green: live cells on hydrogel surface; Red: dead cells (n=3), n.s.: not significant. * p<0.05, ** p<0.01

FIG. 6 shows an animal experiment for evaluating bone formation. (A) Preparation of hydrogels: 7 pieces of sponge used for each hydrogel. (B) Macroscopic images of bone defects and of bone defects treated with a material immediately after surgery (n=4). (C) Microcomputed tomography (μ-CT) images of bone defects after 3 and 6 weeks. (D) Hematoxylin and eosin (H&E) staining of bone defects after 3 and 6 weeks. (E) Quantitative μ-CT analysis after removal of β-TCP particles. Statistical significance: a comparison between particles and hydrogels is indicated as follows: ** p<0.01, BV/TV: bone volume vs. total volume (%).

FIG. 7 shows the degradation of β-tricalcium phosphate (β-TCP) particles in a bone defect. (A) Lateral views of a bone defect after 3 weeks and 6 weeks in μ-CT analysis. The light and dark blue colors in the bone defect indicate residual β-TCP particles. (B) Quantification of residual β-TCP particles using μ-CT analysis. (C) Tartrate-resistant acid phosphatase (TRAP) staining of osteoclasts. (D) Area of a TRAP-stained defect. * p<0.05, ** p<0.01

### DESCRIPTION OF EMBODIMENTS

In the present specification, the amino group is a primary amino group (NH₂) or a secondary amino group (NH), which can form an amide bond with a COOH group of a natural polymer.

The natural polymer is not particularly limited as long as the natural polymer contains (1) a COOH group and (2) at least one member selected from the group consisting of an OH group and an amino group in its repeating unit.

Examples of repeating units having a COOH group include sugar residues having a COOH group, such as glucuronic acid, galacturonic acid, and mannuronic acid, and amino acid residues having a COOH group in the side chain, such as glutamic acid and aspartic acid.

Examples of repeating units having an OH group or an amino group include glucose, mannose, galactose, fructose, fucose, rhamnose, glucosamine, galactosamine, mannosamine, lysine (Lys), ornithine (Orn), and hydroxylysine (Hyl).

Examples of natural polymers include hyaluronic acid, collagen, gelatin, xanthan gum, gellan gum, pectin, pectic acid, alginic acid, a sodium salt of alginic acid, and elastin.

The collagen for use as a raw material for gelatin sponge can be selected from a wide variety of known raw materials for gelatin sponge, including soluble collagen, such as acid-soluble collagen, neutral-salt-soluble collagen, and enzyme-solubilized collagen.

Table 1 below shows the number of amino acids per 1000 amino acid residues (amino acid composition) of typical gelatin.

Table 1 shows gelatin made from pig skin. Gelatin made from cow skin and cow bone has a similar amino acid composition.

Because of the problem of mad cow disease, however, Table 1 shows only the composition of gelatin made from pig skin.

TABLE 1

| Amino Acid | Pig Skin Gelatin |
|---|---|
| Glycine (Cly) | 330 |
| Alanine (Ala) | 112 |
| Valine (Val) | 26 |
| Leucine (Leu) | 24 |
| Isoleucine (Ile) | 10 |
| Serine (Ser) | 35 |
| Threonine (Thr) | 18 |
| Aspartate (Asp) | 45 |
| Glutamine (Gln) | 72 |
| Cysteine (Cys) | 0 |
| Methionine (Met) | 4 |
| Lysine (Lys) | 27 |
| Hydroxylysine (Hyl) | 6 |
| Arginine (Arg) | 49 |
| Histidine (His) | 4 |
| Phenylalanine (Phe) | 13 |
| Tyrosine (Tyr) | 3 |
| Tryptophan (Trp) | 0 |
| Proline (Pro) | 131 |
| Hydroxyproline (Hyp) | 91 |

The gelatin, with Gly accounting for about ⅓, has 117 side-chain COOH groups (Glu+Asp), 86 side-chain amino (NH₂ or NH) groups (Lys+Hyl+Arg+His), and 56 side-chain OH groups (Ser+Thr+Tyr).

The 117 side-chain COOH groups are used in intramolecular or intermolecular crosslinking with the OH groups or amino groups of gelatin via ester bonds or amide bonds, and also used in covalent bonds (ester bonds or amide bonds) or hydrogen bonds with a physiologically active substance containing OH groups or amino groups.

Such crosslinks and covalent bonds via esters or amides can be formed by adding a water-soluble dehydration-condensation agent to water containing gelatin.

The water-soluble dehydration-condensation agent may be added to water containing gelatin and a physiologically active substance. Alternatively, a portion of the water-soluble dehydration-condensation agent may be added to water containing gelatin to form gelatin crosslinks, and then the physiologically active substance and the remaining water-soluble dehydration condensation may be added thereto to bind the physiologically active substance to the gelatin.

In other words, the gelatin crosslinking reaction and the binding reaction of the physiologically active substance may be performed simultaneously, or some degree of gelatin crosslinking may be formed first, and then the physiologically active substance may be allowed to bind (at which time the gelatin crosslinking reaction may also occur simultaneously).

Although gelatin was explained as an example above, functional crosslinked structures can also be obtained with natural polymers other than gelatin by reacting it with a physiologically active substance and a water-soluble dehydration-condensation agent in water in the same manner.

The physiologically active substance is not particularly limited as long as it has an OH group or an amino group.

Examples thereof include catechins (e.g., catechin (C), gallocatechin (GC), epicatechin (EC), epigallocatechin (EGC), catechin gallate (CG), epicatechin gallate (ECG), gallocatechin gallate (GCG), epigallocatechin gallate (EGCG)), polyphenols (e.g., quercetin and derivatives thereof), proanthocyanidins, anthocyanins, flavonoids (e.g., flavonols, flavones, isoflavones, flavanones, and flavanols), soy isoflavones (e.g., daidzein, genistein, glycitein, and their glycosides such as daidzin, genistin, and glycitin, as well as their metabolite, such as equol), and polyamines (e.g., spermine and spermidine).

These physiologically active substances may be used alone or in a combination of two or more. For example, the catechins may be a mixture containing two or more types of catechins, and may also contain components other than catechins depending on the degree of purification.

Catechins that contain EGCG are preferred, and those that contain EGCG as a main component are particularly preferred. A physiologically active substance containing EGCG as a main component may be 100% EGCG.

The physiologically active substance may be reacted in an amount of about 0.0001 to 15 parts by mass, preferably 0.001 to 10 parts by mass, and more preferably 0.01 to 5 parts by mass, per 100 parts by mass of the natural polymer.

The dehydration-condensation agent used in the present invention is not particularly limited as long as it is water-soluble, capable of forming an ester bond or amide bond between a COOH group of a natural polymer and an OH or amino group of a physiologically active substance in an aqueous solution, and capable of crosslinking the natural polymer.

Examples of dehydration-condensation agents include triazine-based condensation agents, such as 4-(4,6-dimethoxy-1,3,5-triazin-2-yl)-4-methylmorpholinium chloride (DMT-MM), a water-soluble salt of 1-cyclohexyl-3-(2-morpholinoethyl)carbodiimide, and 1-ethyl-3-(3-dimethylaminopropyl)carbodiimide hydrochloride (WSC).

These can be used alone or in an appropriate combination.

The water-soluble dehydration-condensation agent may be reacted in an amount of about 1 to 1,000 parts by mass, preferably about 5 to 500 parts by mass, and more preferably about 10 to 100 parts by mass, per 100 parts by mass of the natural polymer.

The dehydration-condensation agent used can be removed by washing with water.

The functional crosslinked structure can be purified by a commonly used method, such as dialysis, filtration, gel filtration chromatography, or ion-exchange chromatography.

The functional crosslinked structure used in the present invention functions as a matrix for retaining β-TCP particles, not only improving ease of handling by limiting the scattering, movement, and leakage of β-TCP particles, but also enhancing the bone regeneration capacity due to β-TCP.

Additionally, promoting the absorption of β-TCP particles can reduce the amount of β-TCP administered, thereby reducing the amount of remaining β-TCP during bone regeneration.

Although β-TCP is necessary for bone regeneration, β-TCP remaining after sufficient progression of bone regeneration may hinder bone regeneration or cause infection.

The bone regeneration material of the present invention is ideal because the absorption of β-TCP particles proceeds simultaneously with the progression of bone regeneration.

The composite of the present invention preferably contains 0.0001 to 99.9999 mass % of the functional crosslinked structure and 0.0001 to 99.9999 mass % of the β-TCP particles, and more preferably contains 5 to 60 mass % of the functional crosslinked structure and 40 to 95 mass % of the β-TCP particles, relative to the solid content of the composite of the present invention.

The composite of the present invention may contain water or an aqueous solution in addition to these solids.

Examples of aqueous solutions include buffer solutions, cell culture media, infusion solutions, and blood (plasma and serum). The bone regeneration material containing an aqueous solution may be in the form of a hydrogel.

The water content in the bone regeneration material is preferably 0.0001 to 99.9999 mass %, and more preferably 0.1 to 99 mass %.

If the bone regeneration material does not contain water, the bone regeneration material applied to or implanted into a site that needs bone regeneration (e.g., a bone defect) may absorb water from body fluids and form a hydrogel. However, it is also possible to have the bone regeneration material absorb a water-containing solvent beforehand and transplant the material in a hydrogel form.

The average particle size of the β-TCP particles is 5 to 5000 μm, and more preferably 150 to 3000 μm. The average particle size can be measured according to a laser diffraction-scattering method (wet method).

The composite of the present invention can be obtained by mixing the functional crosslinked structure and β-TCP particles in a water-containing solvent, lyophilizing the mixture, and further optionally crosslinking the mixture.

Crosslinks after lyophilization are preferably formed by vacuum heating.

The reaction temperature in vacuum heating is preferably 24 to 250° C., and more preferably 80 to 175° C. The reaction time in vacuum heating is preferably 1 minute to 72 hours, and more preferably 1 to 48 hours. The reaction pressure in vacuum heating is preferably atmospheric pressure to absolute vacuum, and more preferably −0.01 to −0.101 MPa.

The composite and bone regeneration material of the present invention may be of any shape that matches the shape of the bone defect to which the composite or material is applied, and may be in the shape of, for example, a sheet, block, disk, bullet, cylinder, or cube. The composite or bone regeneration material for use may a combination of two or more different shapes.

The shape of the composite or bone regeneration material of the present invention can be adjusted during lyophilization or before transplantation so as to fit the shape of the bone defect. The composite or bone regeneration material in a flexible material form such as a hydrogel can also be adjusted at the time of surgery or implantation so as to fit the shape of the bone defect.

### EXAMPLES

The present invention is described below in more detail with reference to Examples.

### Example 1

### 1.1 Preparation of Sponge (Hydrogel Precursor)

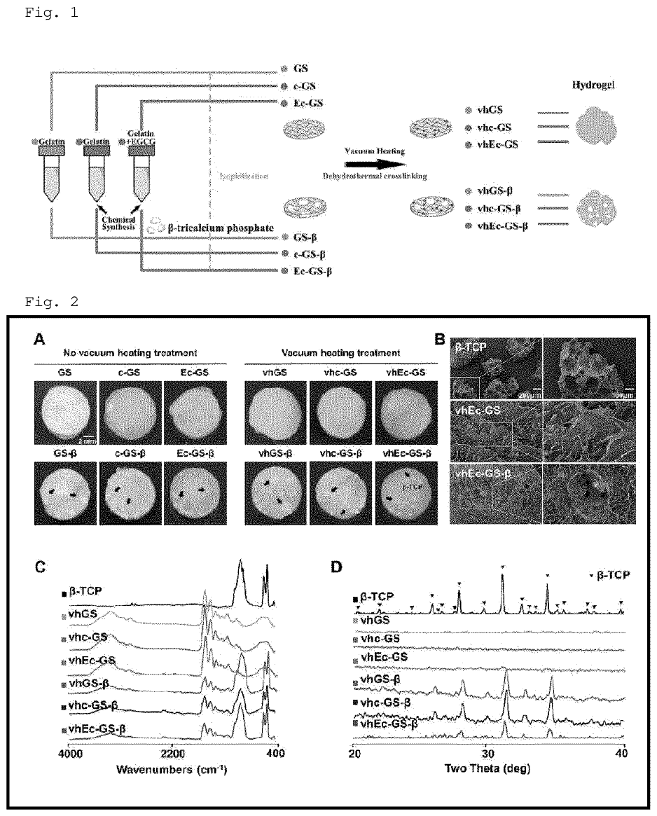

To obtain sponge, which is a precursor of a hydrogel, the following solutions (1) to (3) were prepared:
(1) an untreated gelatin solution (gray),
(2) a chemically crosslinked gelatin solution (blue), and
(3) an EGCG-modified chemically crosslinked gelatin solution (red) (FIG. 1).

A total of six solutions, three containing β-TCP particles and three without β-TCP particles, were prepared and lyophilized to obtain sponges (FIG. 1 and Table 2).

A portion of the obtained sponges was thermally crosslinked by vacuum heating at 150° C. under −0.1 MPa as shown in FIG. 1.

TABLE 2
Summary of prepared sponges.

| | | Gelatin | | β-TCP | Chemical | Vacuum |
|---|---|---|---|---|---|---|
| Sample Name | Abbreviation | (mg) | EGCG (mg) | (mg) | Synthesis | Heating |
| Gelatin sponge | GS | 1 | 0 | 0 | No | No |
| Chemically | c-GS | 1 | 0 | 0 | Yes | No |
| synthesized | | | | | | |
| gelatin sponge | | | | | | |
| Chemically | Ec-GS | 1 | 0.0028 | 0 | Yes | No |
| synthesized | | | | | | |
| gelatin sponge | | | | | | |
| modifi ed with | | | | | | |
| EGCG | | | | | | |
| GS | GS-β | 1 | 0 | 4 | No | No |
| incorporating | | | | | | |
| β-TCP | | | | | | |
| c-GS | c-GS-β | 1 | 0 | 4 | Yes | No |
| incorporating | | | | | | |
| β-TCP | | | | | | |
| Ec-GS | Ec-GS-β | 1 | 0.0028 | 4 | Yes | No |
| incorporating | | | | | | |
| β-TCP | | | | | | |
| Vacuum-heated | vhGS | 1 | 0 | 0 | No | Yes |
| GS | | | | | | |
| Vacuum-heated | vhc-GS | 1 | 0 | 0 | Yes | Yes |
| c-GS | | | | | | |
| Vacuum-heated | vhEc-GS | 1 | 0.0028 | 0 | Yes | Yes |
| Ec-GS | | | | | | |
| Vacuum-heated | vhGS-β | 1 | 0 | 4 | No | Yes |
| GS-β | | | | | | |
| Vacuum-heated | vhc-GS-β | 1 | 0 | 4 | Yes | Yes |
| c-GS-β | | | | | | |
| Vacuum-heated | vhEc-GS-β | 1 | 0.0028 | 4 | Yes | Yes |
| Ec-GS-β | | | | | | |

A gelatin (1%) solution was prepared by dissolving 100 mg of RM-Gelatin (JELLICE) in 10 mL of ultrapure water at 50° C.

A solution of chemically crosslinked gelatin and a solution of EGCG-modified chemically crosslinked gelatin were prepared according to a known method described in literature (Int. J. Mol. Sci. 2015, 16, 14143-14157; Molecules 2018, 23, 876).

EGCG (purity: 90%) was kindly provided by Dr. Yukihiko Hara (Tea Solutions, Hara Office, Inc.).

A 9 mm hole was made in a PTFE sheet (Misumi Group, Inc., Tokyo, Japan), and the bottom was covered with a polydimethylsiloxane sheet made of SILPOT 184 (Dow Corning Toray Co., Ltd., Tokyo, Japan).

To produce sponges, a solution of 100 μL of gelatin, a solution of chemically crosslinked gelatin, or a solution of EGCG-modified chemically crosslinked gelatin was added to the hole with or without 4 mg of 3-TCP particles (500-1000 μm, CatalyMedic, Inc.), and the sheet was placed in a MediFridge (Fukushima Galilei Co., Ltd., Kagoshima, Japan) at −20° C.

The frozen material was then lyophilized in DC800 (Yamato Scientific Co., Ltd., Tokyo, Japan) for 24 hours to obtain a sponge material.

Vacuum heating was performed at 150° C. for 24 hours by using AVO-250NS (AS ONE Co., Ltd., Osaka, Japan) and DA-20D (Ulvac Kiko, Inc., Kanagawa, Japan) under a gauge pressure of −0.1 MPa.

Macrophotographs were taken with an EOS 600D digital camera (Canon, Inc.).

A coating process was performed for 3 seconds using an osmium coater (Vacuum Device Co., Ltd., Ibaraki, Japan), and the materials were observed with an electron microscope. The presence of β-TCP particles was confirmed by using a field emission scanning electron microscope (FE-SEM; S-4800; Hitachi, Ltd., Tokyo, Japan) at 5 kV.

Spectra were obtained according to attenuated total reflectance Fourier-transform infrared (ATR-FTIR) spectroscopy (IRAffinity-1S; Shimadzu Co., Kyoto, Japan) at a wavenumber of 400 to 3900 cm⁻¹.

Spectra were measured at 200 to 400 according to an X-ray diffraction (XRD) method (XRD-6000; Shimadzu Co.).

1.2. Test of Leakage of β-TCP Particles from Hydrogel

The leakage rate of β-TCP particles from a hydrogel was measured in a small test tube using a piece of flaky sponge and 1000 μL of ultrapure water.

The samples were placed in a refrigerator at 4° C. for 24, 48, and 72 hours.

After incubation for a predetermined period of time, matter other than the leaked calcium phosphate (CaP) was removed, and the remaining CaP was dried at 55° C. for 24 hours in a drying oven (DV 400; Yamato Scientific Co., Ltd.).

The dispersed β-TCP particles were weighed with an electronic balance (GR-202; Misumi Group, Inc.), and the percentage by mass of the dispersed particles was calculated using the formula: β-TCP (mg)/4×100%.

To confirm the structural stability of the hydrogels, gelatin was immunostained with FDV-0035 (10 μg/mL; Funakoshi Co., Ltd., Tokyo, Japan) and a Qdot 655 streptavidin complex (1 pig/mL, Thermo Fisher Scientific, Inc., Waltham, MA, USA), which are staining solutions, according to the manufacturer's instructions.

The time required for a hydrogel was tested by adding 70 μL of a water droplet to each sponge using a wettability evaluation device (LSE-ME2; Nick Corporation, Saitama, Japan).

1.3. Analysis of Biocompatibility of Hydrogel UMR106 cells (American Type Culture Collection, Manassas, VA, USA) at the 7th passage were cultured in a 96-well plate (AGC Techno Glass Co., Ltd., Shizuoka, Japan) with 5,000 cells per well for 3 days.

The culture medium used was Dulbecco's modified Eagle's medium (DMEM) (MilliporeSigma, Burlington, MA, USA) supplemented with 10% fetal bovine serum and 1% antibiotics.

Subsequently, one disk of
a hydrogel containing a vacuum heat-treated gelatin sponge (vhGS) hydrogel,
vacuum heat-treated and chemically synthesized gelatin sponge (vhc-GS),
vacuum heat-treated and chemically synthesized gelatin sponge conjugated with EGCG (vhEc-GS),
β-TCP particle-introduced vhGS (vhGS-β),
β-TCP particle-introduced vhc-GS (vhc-GS-β), or
β-TCP particle-introduced vhEc-GS (vhEc-GS-β) and DMEM was added to the wells.

The well plates were placed in an incubator.

The number of viable cells in the well-plates was counted after 24, 48, and 72 hours of culture using a Cell Counting Kit-8 (CCK-8; Dojindo Laboratories, Tokyo, Japan).

An LIVE/DEAD Viability/Cytotoxicity Kit for Mammalian Cells Protocol was obtained from Thermo Fisher Scientific, Inc.

UMR106 cells at the 7th passage were seeded onto the hydrogels (20,000 cells/mL), incubated for 24 hours, and observed with a fluorescent cell imager (Bio-Rad Laboratories, Inc., Hercules, CA, USA).

The areas occupied by green and red were measured with ImageJ (Java 1.8.0_172; National Institutes of Health, Bethesda, MD, USA).

The percentage of dead cells was determined using the following formula:

$$Red area / ( red area + green area ) \times 100 \% ( n = 3 ) .$$

### 1.4. Animal Experiment

The experiment was approved by the Ethics Committee of Osaka Dental University under Animal Experiment Approval Number 2102017.

Eight-week-old SD rats were purchased from Shimizu Laboratory Supplies Co. (Kyoto, Japan), and models with a 9 mm critical-sized calvarial bone defect were prepared.

For a control sham surgery group, the defect was treated with 100 μL DMEM (without particles or a hydrogel).

For an experimental group, β-TCP particles (12 or 28 mg) or a hydrogel containing the same amount of β-TCP particles was implanted.

Integrated hydrogels were each prepared from seven pieces of sponge with 100 μL medium, specifically from vhEc-GS-β (12), which is a mixture of four pieces of sponge (vhEc-GS) and three pieces of sponge (vhEc-GS-β (4 mg β-TCP)), and from vhEc-GS-β (28), composed of seven pieces of sponge (vhEc-GS-β (4 mg β-TCP)).

Four rats were used in each group at 3 and 6 weeks.

Samples were collected by cardiac perfusion fixation after 3 or 6 weeks.

Tissues were fixed using 10% neutral buffered formalin (Wako Chemicals USA, Inc., Richmond, VA, USA).

Microcomputed tomography (μ-CT) analysis was performed with a Skyscan 1275 (Bruker Co., Billerica, MA, USA) at 80 μA and 70 kV.

The samples were decalcified by immersion in decalcifying solution B (Wako Chemicals USA, Inc.) for 5 days.

The tissues were then sectioned according to the Kawamoto method (Arch. Histol. Cytol. 2003, 66, 123-143).

Sections were prepared so as to have a thickness of 6 μm by using a Leica CM1950 (Leica Biosystems, Wetzlar, Germany).

A hematoxylin and eosin (H&E) staining solution was purchased from Muto Pure Chemicals Co., Ltd. (Tokyo, Japan).

The emergence of osteoclasts was evaluated using a tartrate-resistant acid phosphatase (TRAP) staining kit (Wako Chemicals USA, Inc.) according to the procedure described in the instruction manual.

The amount of osteoclasts was calculated using the following formula:

$$purple - stained area on each photograph / total image area \times 100 \% ( n = 3 )$$

### 1.5 Statistical Analysis

Prism 9.0.0 software (GraphPad Software, San Diego, CA, USA) was used for data processing and analysis in this study.

All statistical analyses were performed according to the one-way ANOVA and Tukey's multiple comparisons test, and values were expressed as mean±standard deviation.

p<0.01 or 0.05 indicates a significant difference.

### 2. Results

### 2.1. Characterization of Sponge (Hydrogel Precursor)

Macroscopic observation found no significant changes before and after vacuum heating treatment.

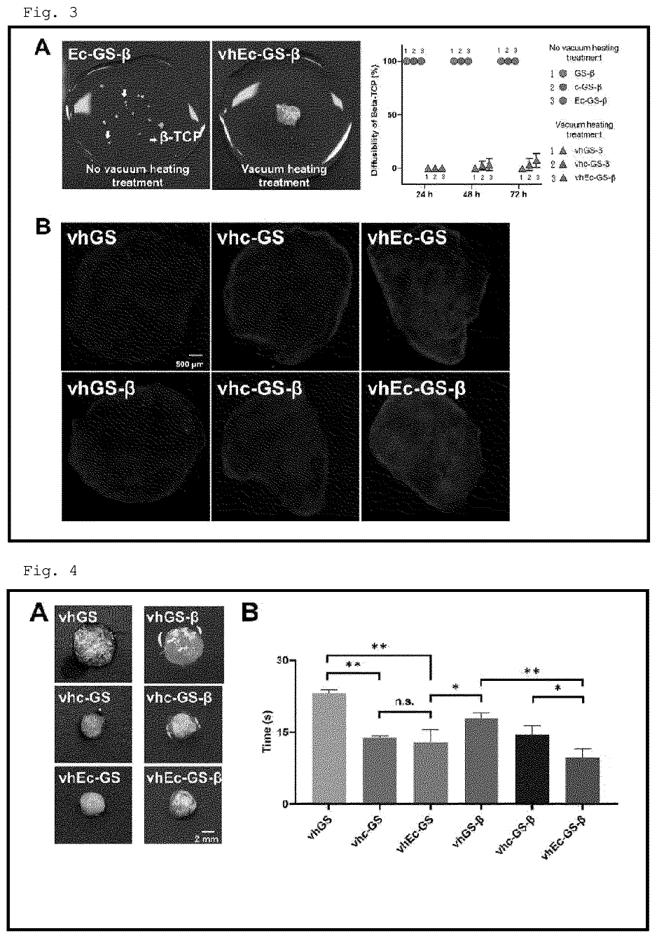

FE-SEM observation found that the porous β-TCP particles were well encapsulated in all sponges (FIGS. 2A and 2B).

The ATR-FTIR and XRD results indicated that the presence of gelatin and changes in the crystalline phase of β-TCP particles were negligible (FIGS. 2C and 2D).

After characterization of the sponges, hydrogels were prepared by adding pure water or medium in the following experiments.

2.2. Leakage of β-TCP Particles from Hydrogel

After hydrogels were prepared by adding pure water to each sponge, the gels were immersed in an aqueous solution and gently shaken to evaluate leakage of β-TCP particles.

Hydrogels that were not thermally crosslinked quickly collapsed in liquid, allowing the β-TCP particles to outflow, whereas thermally crosslinked hydrogels maintained their morphology and retained the β-TCP particles even after 72 hours (FIG. 3A).

Even after gelatin staining with repeated washing, all samples that underwent the thermal crosslinking process tightly retained the morphology as hydrogels (FIG. 3B).

These results indicate that thermal crosslinking enhances the stability of hydrogels and their retention of β-TCP particles.

### 2.3. Hydrogel Morphology and Water Absorption Rate

The hydrogel-forming process can affect operability.

The water absorption rate of a material is directly related to specimen preparation time.

After ultrapure water was added, vhc-GS or vhEc-GS, and materials containing β-TCP particles in addition to vhc-GS or vhEc-GS significantly shrank, whereas vhGS and vhGS-β relatively maintained their morphology (FIG. 4A).

The water absorption rate of vhEc-GS and vhEc-GS-β was significantly faster than that of vhGS and vhGS-β (FIG. 4B).

### 2.4. In Vitro Biocompatibility Testing

The sponges were formed into hydrogels with DMEM before use in a cell culture test.

Two different methods were used to evaluate the cytotoxicity of the hydrogels by using the UMR106 rat osteoblastic cell line.

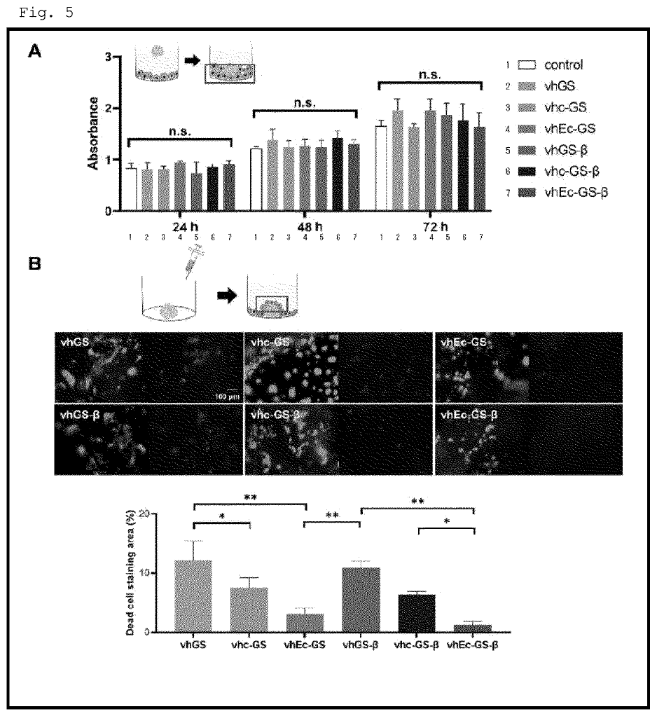

The results of a CCK-8 assay for cells that were not brought into contact with hydrogels indicated no noticeable cytotoxicity (FIG. 5A).

Stained live or dead cells that had been in contact with the hydrogel derived from vh-GS or vh-GS-β were observed.

The number of dead cells was significantly low in the case of the hydrogel derived from vhEc-GS or vhEc-GS-β.

### 2.5. Bone Regeneration and β-TCP Particle Absorption

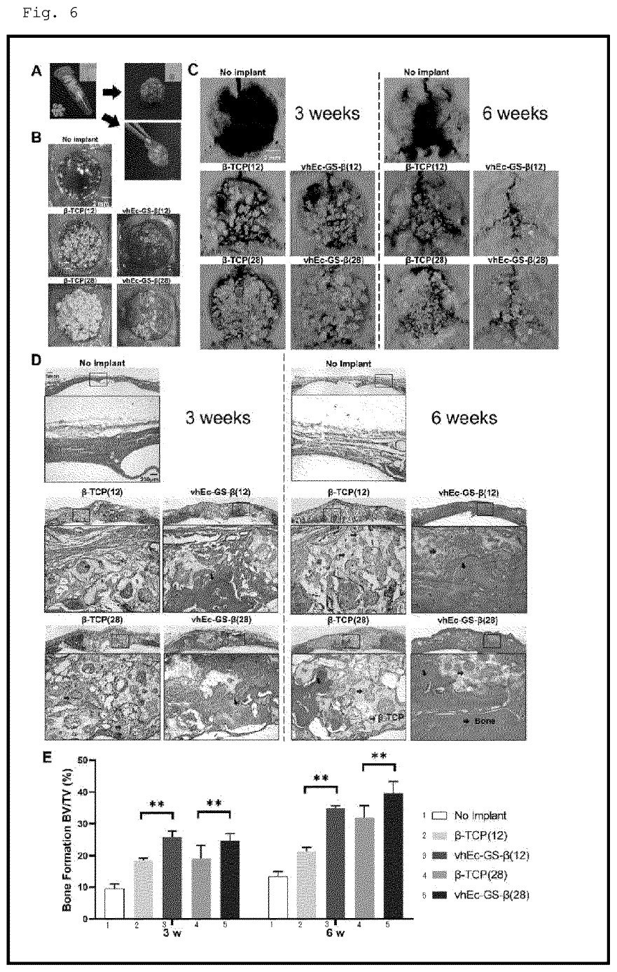

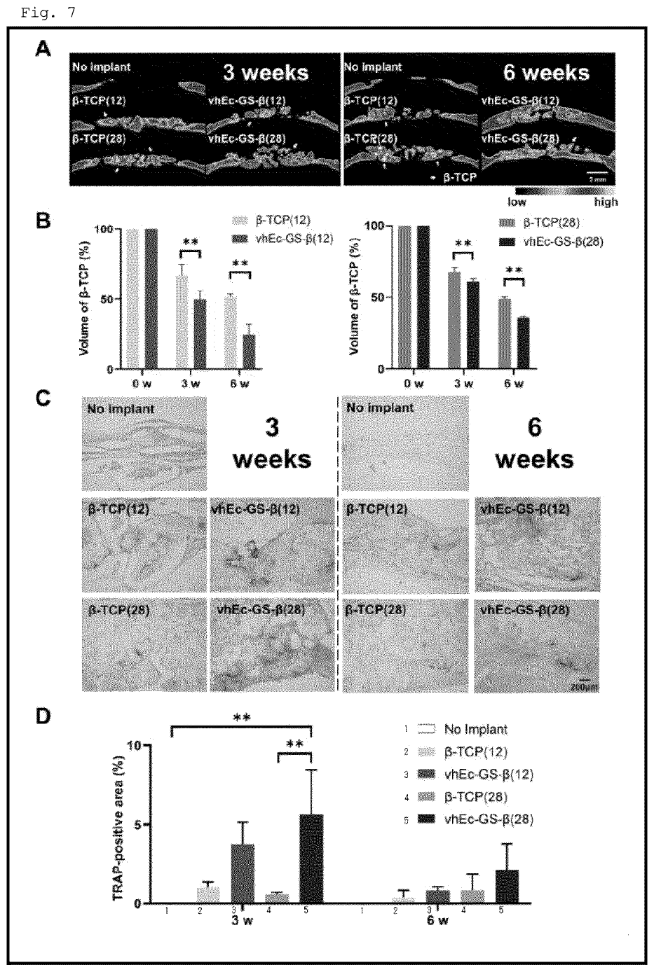

Considering the advantages of vhEc-GS-β in terms of operability and cytocompatibility over particles alone, the osteogenic capability of vhEc-GS-β-derived hydrogel and β-TCP particles and the absorption of β-TCP particles were compared using a critical-sized rat calvarial bone defect (9 mm) (FIG. 6).

Seven pieces of vhEc-GS-D (containing 28 mg of β-TCP) or a combination of four pieces of vhEc-GS and three pieces of vhEc-GS-β (containing 12 mg of β-TCP) according to the size of the bone defect was mixed with DMEM and integrated to prepare hydrogels, which were then implanted (FIGS. 6A and 6B).

The hydrogels were easily operable and could be pinched with tweezers (FIG. 6A).

The amount of β-TCP particles was standardized to 12 mg or 28 mg per defect (the number in parentheses is the mass of the β-TCP particles).

The μ-CT results showed increased opacity in the hydrogel derived from the vhEc-GS-β sponge as compared with β-TCP particles alone at 3 and 6 weeks after implantation (FIG. 6C).

H&E staining images confirmed an increase in the area of new bone (FIG. 6D).

Furthermore, μ-CT morphometric analysis after removal of the opaque images derived from β-TCP particles indicated that vhEc-GS-β(12) and vhEc-GS-β (28) formed bone significantly more than β-TCP particles alone (FIG. 6E).

### 2.6. β-TCP Absorption

The lateral views of the bone defect in μ-CT images show that light and dark blue particles (indicating β-TCP) were present more abundantly in the defect of the β-TCP group than in the defect treated with the vhEc-GS-β-derived hydrogel at 6 weeks (FIG. 7A).

The β-TCP particles in the vhEc-GS-β group were absorbed more rapidly than those in the β-TCP group (FIGS. 7A and 7B).

TRAP staining, which shows osteoclasts, indicated that the number of osteoclasts was increased in the vhEc-GS-β (28) group compared with that in the group to which 28 mg of 3-TCP was administered (FIGS. 7C and 7D).

1. A composite comprising
a matrix, and
β-tricalcium phosphate (β-TCP) particles dispersed in the matrix,
the matrix comprising a functional crosslinked structure wherein
a natural polymer having at least one member selected from the group consisting of an OH group and an amino group, and a COOH group in a repeating unit is bound to a physiologically active substance having an OH group or an amino group via an ester bond, an amide bond, or a hydrogen bond, and
the COOH group of the natural polymer is crosslinked with the at least one member selected from the group consisting of an OH group and an amino group of the natural polymer.

2. The composite according to claim 1, wherein the physiologically active substance is at least one member selected from the group consisting of catechins, polyphenols, proanthocyanidins, anthocyanins, flavonoids, soy isoflavones, and polyamines.

3. The composite according to claim 1, wherein the natural polymer is selected from the group consisting of hyaluronic acid, collagen, gelatin, xanthan gum, gellan gum, pectin, pectic acid, alginic acid, a sodium salt of alginic acid, and elastin.

4. The composite according to claim 1, wherein the matrix is a gel.

5. The complex according to claim 1,
wherein the physiologically active substance is a catechin that mainly contains epigallocatechin gallate (EGCG), and
the natural polymer is gelatin.

6. A method for producing a composite of a functional crosslinked structure and pi-TCP particles,
the method comprising
reacting a natural polymer having at least one member selected from the group consisting of an OH group and an amino group, and a COOH group in a repeating unit with a physiologically active substance having an OH group or an amino group,
in water, and in the presence of a water-soluble dehydration-condensation agent,
to bind the COOH group of the natural polymer to the physiologically active substance via an ester bond, an amide bond, or a hydrogen bond, and
to crosslink the natural polymer,
thereby obtaining a functional crosslinked structure, and
mixing a solution of the obtained functional crosslinked structure with β-tricalcium phosphate (β-TCP) particles, and
subjecting the mixture to lyophilization and vacuum heating.

7. The method according to claim 6, further allowing a water-containing solution to act to the obtained composite
to obtain a gelled composite.

8. A bone regeneration material comprising the composite of any-ene claim 1.
